(19) United States
(12) Patent Application Publication
(10) Pub. No.: US 2004/0157867 A1
(43) Pub. Date: Aug. 12, 2004

(54) Pyrimidine phosphorylase as a target for imaging and therapy

(76) Inventors: Raymond W. Klecker, Silver Spring, MD (US); Jerry M. Collins, Rockville, MD (US)

Correspondence Address: VENABLE, BAETJER, HOWARD AND CIVILETTI, LLP, P.O. BOX 34385, WASHINGTON, DC, 20043-9998, US

(21) Appl. No.: 10/466,423

(22) PCT Filed:
(86) PCT No.: PCT/US02/01216

(51) Int. Cl. A61K 51/00; A61K 31/513; C07D 239/02

(52) U.S. Cl. 514/269; 424/1.11; 544/309

(57) ABSTRACT

Thymine analogs and methods for their use as diagnostic and therapeutic agents for tumors.

### FIELD OF THE INVENTION

The invention relates to thymine analogs; to their use at tracer doses as diagnostic tools; and a treatment for tumors at higher doses.

### BACKGROUND OF THE INVENTION

Thymidine Phosphorylase (TPase; EC 2.4.2.4) is a well-known enzyme for pyrimidine catabolism. Recently it has been found to be associated with angiogenesis in tumors. Angiogenesis is the production of new blood vessels; tumors must stimulate that production to support new growth. A large series of growth factors (e.g., VEGF, EGF, FGF, TNF, PDEC-GF) and their receptors are implicated in angiogenesis. The elucidation of their roles and interrelationships is the subject of extensive research. Platelet derived endothelial cell growth factor (PDECGF) has recently been identified as TPase (Usuki, 1992; van Triest, 2000).

Expression of TPase in tumors has been linked to clinical outcome for many tumor types. High expression of TPase is associated with an extremely poor overall survival rate compared with low TPase. These findings are illustrated in FIG. 1. Data from three different countries (the Netherlands, England, and Japan) on 4 different cancer types: colorectal, head/neck, bladder, and cervical are included (van Triest, 2000, Koukourakis, 2000, Arima, 2000, Fujimoto, 1999).

Angiogenesis is considered to be one of the most important processes in the development of tumors; the focus of much current research is on developing anticancer therapeutics targeting angiogenesis. The mechanism of the association of TPase and angiogenesis is not currently known. Current approaches to determine the effect of an antitumor drug on its specific target are generally invasive; i.e., require obtaining a biopsy from the patient. Such studies are possible for TPase or other factors related to angiogenesis, measuring factors such as blood vessel density in the biopsy, or analysis for specific growth factor targets. However, in addition to the discomfort and/or risks associated with biopsy procedures, only a small sample of tissue is obtained, which may not be representative of the entire region. Moreover, a time course of serial biopsies is not practical. The success rate for obtaining even a single biopsy in clinical studies is poor, and presents a major hurdle for contemporary approaches to molecular assessment of treatment.

Although a number of anti-angiogenesis drugs are now being tested in-patients with cancer, it has been difficult to evaluate their success. It seems likely that inhibiting angiogenesis would inhibit tumor growth, with classic clinical endpoints (e.g., survival), but trials of these drugs would be long, cumbersome, and expensive. The most frequently used intermediate measure for cytotoxic drugs, tumor shrinkage, may not be useful for anti-angiogenic agents.

### SUMMARY OF THE INVENTION

A goal of the present invention is to provide a non-invasive means of measuring the efficacy of anti-angiogenic agents. Non-invasive, external imaging methods avoid the need for biopsies, and also have the capability of scanning large areas of the body, the entire body if necessary. Multiple time points can be obtained during a single imaging session, and longitudinal evaluations (before and after therapy) are possible. The ability to image factors such as TPase that are associated with angiogenesis could have enormous potential as a guide to therapeutic development, as well as in the treatment of patients. Additionally, changes with angiogenesis are implicated in arthritis, psoriasis, female reproductive cycling, wound healing, and overgrowth of blood vessels in the retina, as a consequence of diabetes. It is not presently known whether TPase is a biomarker of treatment success. This information is important in determining whether TPase is actually an angiogenic factor, or if it is a surrogate marker for angiogenic activity.

The present invention solves the problems of the previous methods by noninvasive, external imaging methods based on TPase levels. TPase catalyses the reaction cleaving thymidine (dThd) to form thymine (Thy) and deoxyribose-1-phosphate (dR-P) via the following reaction as well as the reverse reaction:

dThd+Pᵢ→Thy+dR-P

The reverse reaction is more useful in the present invention for trapping thymidine-based probes within the tumor, because thymine readily leaves the cell at the end of the reaction. Nucleosides, such as thymidine, are efficiently phosphorylated within the cell; thymine molecules when converted to thymidine by TPase will remain trapped within the cell, either as nucleotides or via subsequent processing and incorporation as DNA When the reaction products are trapped intracellularly, the accumulation within cells will be proportional to the rate of conversion by TPase.

Thy+dR-P→dThd

dThd→dTMP→→DNA

Tumor cells with high levels of TPase expression convert more thymine than cells with lower levels of TPase expression.

Along with thymine itself, a variety of thymine analogs are substrates for TPase. In general, suitable thymine analogs include any with substitution at the 5-position of the uracil base. In addition to thymine, such thymine analogs can include, without limitation, 5-Br-Ura, 5-I-Ura, and 5-CF₃-Ura (trifluorothymine). Uracil and 5-FUra are also substrates for TPase, but are less suitable to the current application because they are also substrates for Uridine Phosphorylase (UPase). It would not be possible to separate the contributions of TPase and UPase as determinants for intracellular accumulation of Ura and 5-FUra.

The following is the structure of 5-X-Ura (thymine and its analogs), where X═CH₃, Br, I, CF₃.

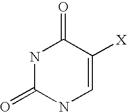

Hereinafter, compounds of that formula may be designated as 5-X-Ura compounds. As can be seen from the formula, X is on the 5 position in the 6-member ring. Substrate metabolism must yield identifiable product in order to measure conversion. The method of the present invention is to add a detection molecule to the substrate so as to produce a measurable product. The detection molecule can be any marker known in the art, including, in a preferred embodiment, any radiolabel containing reagent. By radiolabeling a thymine analog with a positron emitter, the probe has the potential to non-invasively monitor the localization of TPase within the tumor and/or normal host tissue by use of imaging technologies. A preferred technique is to use a PET (Positron Emission Tomograph) scanner. Gamma scintigraphy and single-photon imaging (SPECT) are also suitable methods, but these technologies either provide planar images or lower resolution. Because PET imaging can provide 3-dimensional information with high sensitivity, positron-emitters are particularly desirable for imaging probes.

Positron-emitting isotopes have a variety of half-lives: ¹²⁴⁻Iodine, 4 days; ⁷⁶⁻Bromine, 16 hours; 18−Fluorine, about 2 hours, and 11−Carbon, 20 minutes. This range of physical half-lives allows the isotope to be matched to the underlying biochemical process. For phenomena that occur on a time scale of an hour or less, the short half-life of ¹¹C, 20 minutes, provides rapid elimination from the body, thereby permitting serial “snapshots” of changes in tumor activity. For phenomena that extend over a period of a day, [⁷⁶Br]-BrUra would be more accurate because its physical half-life is 16 hours.

2-[¹¹C]-Thy has been directly administered to human beings, as part of a study to model the kinetic behavior of [¹¹C]-thymidine (Mankoff, 1999). The subjects were healthy volunteers, and no assessment of tumor-specific parameters was possible. In addition, many patients have been exposed to [¹¹C]-thymine as a metabolite when [¹¹C]-thymidine was administered.

A Positron-Emitting Compound Having the Formula (“5-X-Ura”)

The present invention encompasses a method of synthesizing compounds that are substrates for TPase which contain detection markers. As discussed above, 5-X-Ura is class of substrates for the reverse reaction of TPase. A method of making [¹¹C]—CH₃-Ura, thymine has been published (Mankoff, 1999). Methods of synthesis of several members of this series are disclosed in Example 1. Other members of the group can be synthesized using similar methods.

Evaluation of TPase Activity in Tumor or Other Body Sites

In another aspect the present invention encompasses a method of measuring relative TPase levels in-patients or experimental subjects using labeled TPase substrates. Following injection of the positron-labeled probe into a patient, cells with high TPase will trap the probe at a higher level than cells with low TPase. Images from such patients, with standard PET scanner techniques, will reveal the areas of high radioactivity and, thus, areas of high TPase activity. This method can both characterize known tumors as to their TPase activity and localize possible metastatic sites in patients with angiogenic tumors. TPase levels are related to clinical outcome in many tumor types irrespective of angiogenesis, making it a useful target for imaging. It can also detect high TPase activity in other disease states characterized by angiogenesis.

By “elevated TPase level” (high TPase activity) is meant any cell or tissue in an individual in which TPase activities are raised above the normal range found in that individual, or within the general population, or within the particular population of cells or tissue being measured. In other words, an individual need not have a high overall level of TPase activity, but one which may be indicative of a tumor or other abnormal condition at the site where the TPase level is measured. By “therapeutically effective amount” is meant an amount of a pharmaceutical substance that is effective in reducing the level of TPase activity, and/or in decreasing the rate of angiogenesis, or the growth rate of a tumor or progress of any pathological condition associated with angiogenesis.

Although DNA synthesis is a requirement for efficient trapping, the data in Example 2 (below) demonstrate that labeled thymine (and its analogs) does not function as a probe for cellular proliferation, as might have been thought. 5-X-Ura localization inside cells is related to TPase activity, but independent from cellular proliferation rates. The five, human-derived, tumor cell lines used in this study all had identical proliferation rates (doubling times of about 24 hours), but the incorporation of thymine or bromouracil varied more than 10-fold, and was directly associated with the amount of TPase in each cell line.

Evaluation of Anti-Angiogenesis Therapy

The present invention can also be used for evaluating the effectiveness of TPase inhibitors in a tumor or other body sites. The search for angiogenesis inhibitors is an important area of research for oncology and other medical conditions. The present invention allows single TPase measurement and serial measurements before and during treatment with proposed TPase inhibitors.

Imaging of TPase levels can be used to evaluate the effects of chemotherapy on tumors. Monitoring TPase provides an independent method of evaluating anti-angiogenic drugs. Based on of tumor shrinkage, it can take a long period of treatment to determine whether chemotherapy has a beneficial effect on the tumor. By contrast, PET imaging with the probes of the present invention provides an immediate assessment of effectiveness at the target. A clinical practitioner could rapidly individualize treatment of a condition characterized by angiogenesis.

Several experimental inhibitors of TPase have been assessed to determine their effectiveness as potential therapeutic agents, these include acyclothymine and 6-amino-thymine. Such inhibitors have been found effective in isolated enzyme preparations. “TPI”, 5-Chloro-6-(2-iminopyrrolidin-1-yl)methyl-2,4(1H,3H)-pyrimidinedione, produced average decreases in TPase for the whole body in mice and monkeys, as demonstrated by an increase in plasma concentrations when a thyridine analog (trifluridine, 5-CF₃-dUrd) was administered (Fukishima, 2000, Hamada, 1993, Matthes, 1974). A difficulty in developing this line of anti-tumor therapeutic agents is the problem of evaluation. The only way to determine the effectiveness of TPase inhibitors is by multiple biopsies. Use of the present invention addresses this problem. PET imaging with the probe molecules disclosed herein permits evaluation of the 3-dimensional distribution of TPase activity throughout the body at times before, during or after therapy. Comparison of TPase activity before and after administration of a TPase inhibitor yields an assessment of the effectiveness of TPase inhibition at any location in the body, and evaluated over a series of time points.

Other therapeutic interventions also effect TPase activity; some interventions produce induction or upregulation of TPase activity, e.g., interferon (akower, 1997); taxanes (Sawada. 1998); cyclophosphamide (Endo, 1999); radiation therapy (Sawada, 1999). These interventions have been demonstrated to affect TPase in cell culture or in animals, but not yet in patients. The use of PET imaging with the probe molecules of the present invention permits evaluation of the 3-dimensional distribution of TPase activity throughout the body, and specifically at its intended target, the tumor. Comparison of TPase activity before and after administration of a TPase inducer/upregulator yields an assessment of the effectiveness of TPase induction/upregulation at any location in the body, and over a series of time points. As discussed, biopsies are an unworkable approach to obtaining such an assessment.

Method of Evaluating the Effectiveness of TPase as a Localized Activator of Drugs

Capecitabine is an example of an anti-cancer prodrug that was designed to be activated by TPase in the tumor (Shimma, 2000; Schuller, 2000). TPase catalyzes the final step for the conversion of capecitabine to fluorouracil. Thus, monitoring expression of TPase activity in the tumor can predict the response of the tumor to capecitabine. Further, in conjunction with the methods of monitoring induction/upregulation described below, the optimal timing of when to administer capecitabine or other similar drug can be determined in the event of fluctuations in TPase activity, as determined by the method this invention.

Method for Selecting Tumors Likely to Benefit from Therapy with 5-X-Ura Compounds

In another embodiment, the 5-X-Ura compounds of the present invention can be used as therapeutic compounds. Tumors with high expression of TPase are also likely to be effected by the 5-X-Ura compounds described. Such tumors are first localized and characterized by the method of the present invention. For imaging, the preferred isotope of 5-X-Ura is a positron emitter, which converts its energy into a photon that leaves the body and can be detected externally. The tumors can then be treated with compounds of the present invention. For therapy, the desired isotope in 5-X-Ura is one that stays localized and exerts its impact over a small distance. As an example, ¹²⁴I is a positron emitter for imaging, and ¹²⁵I is an isotope that can be used for therapeutic applications. The positron-emitting compound used for imaging is administered in trace quantities. The non-radioactive version of the same compound could be used at high concentrations to produce cytotoxicity in cells with high TPase levels. Radioactive isotopes that are used for such therapeutic purposes may be referred to as therapeutic isotopes.

Localization within tumors of drugs or probe molecules is determined by molecular structure, and is unaffected by the specific isotope of an individual atom that comprises the molecule. In a preferred embodiment, the present invention uses imaging to determine if a labeled molecule is localized to a tumor, followed by therapy with the same 5-X-Ura if there is a positive finding. If imaging studies determine that the molecule is not localized to the tumor, the patient is spared the toxicity of chemotherapeutic and/or radio-therapeutic agents.

Method of Treating Tumors with 5-X-Ura Compounds

The present invention encompasses a method of using a non-radioactive compound of the formula, 5-X-Ura, where X is selected from the group of CH₃, CF₃, I, or Br, for the treatment of tumors or other disease associated with high levels of expression of TPase. This family of non-radioactive molecules is known in the art. Structures and synthetic methods have been reported. However, the molecules have been assumed to be pharmacologically inert, it is an insight of the present invention that such molecules could have activity in the treatment of tumors. Example 7 shows that tumor growth is reduced by 5-X-Ura. As therapeutic agents the compounds would be administered in gram quantities, whereas as tracers the dose would be micrograms. Persons of skill in the art will be able to determine suitable dosages from the general knowledge in the art and through routine experimentation.

In another aspect, X, in the compound 5-X-Ura, is selected from the therapeutic isotopes (include alpha and beta emitters) of the atoms in X, including [²¹¹At], [125I], [131I], [⁸²Br]. Generally, pyrimidine bases are considered to be inactive catabolites of the corresponding (deoxyribo) nucleosides. Methods of synthesis of these compounds are found in the Examples. Radioactive compounds in the method of the present invention are used to treat tumors or other diseases associated with high levels of TPase expression (See Example 1.) There are at least two reasons to prefer the radioactive forms over the non-radioactive forms as therapeutic agents: first, greater potency. The radioisotopes provide biophysical modes of cell killing that are more effective than the biochemical modes associated with the non-radioactive molecules. Secondly, for some alpha particles, such as the ones generated by the astatine isotope [²¹¹At], the area of tissue toxicity produced by the radioisotope extends beyond the borders of the cell in which it is localized. This “bystander effect” allows killing tumor cells that are quiescent at the time of exposure (Vaidyanathan, 1996).

Use of a Second Therapeutic Agent to Increase Efficacy Of 5-X-Ura

In another aspect, the 5-X-Ura, therapeutic compounds can be combined with an inhibitor of DPDase to enhance their effect (diagnostic or therapeutic). Dihydropyrimidine dehydrogenase (DPDase) is the endogenous enzyme that rapidly catabolizes the 5-X-Ura series of compounds and also 5-F-Ura and Ura. Thus, there is relatively low systemic exposure to the administered compound, and large exposure to catabolites. For the non-radioactive forms of 5-X-Ura, it may be possible to overcome this catabolism by increasing the dose of 5-X-Ura. However, because DPDase is a highly active enzyme, the required dose increase is 10-100 fold. For the radioactive forms of 5-X-Ura, radio-dosimetry is a major consideration. The risk of radiation damage to normal tissues is determined from total radioactive exposure, i.e., the sum of unchanged radiolabeled 5-X-Ura and all of its labeled catabolites. Thus, the maximum dose of radiolabeled 5-X-Ura that can be administered is limited by the radioactivity contributed by its catabolites. Also, PET imaging is based on total radioactivity, so that radiolabeled catabolites add background “noise” to the images.

Inhibiting systematic catabolism of 5-X-Ura by DPDase reduces labeled catabolites and thereby permits higher concentrations of the parent molecule to be delivered to the target. For a fixed dose of 5-X-Ura, combination with a DPDase inhibitor can permit up to 100-fold increase in parent molecule concentration, producing an increase in the sensitivity of the imaging technique or delivery of cytotoxic treatment. There are several compounds known to inhibit DPDase, including two that have been evaluated in human trials: sorivudine and eniluracil. Sorivudine's inhibition of DPDase was unanticipated and not favorable for its application as an anti-herpes drug. Sorivudine is not currently being developed. Eniluracil was specifically selected to inhibit DPDase catabolism of 5-fluorouracil. Comparative studies demonstrate a 50- to 100-fold increase in 5-FUra exposure in the presence and absence of eniluracil. Baker et al. (2000) report that 2 mg/day per square meter of body surface of 5-FUra is tolerated with eniluracil upon continuous dosing, whereas without eniluracil, 300 mg/day is the standard dose. Plasma clearance of 5-FUra was 0.1 L/min with eniluracil, compared with 5.0 L/min without eniluracil. Because 77% was recovered unchanged in the urine with eniluracil versus less than 10% without eniluracil, there was a dramatic decrease in circulating catabolites. Thus, inhibition of DPDase is an efficient method of increasing circulating levels of 5-X-Ura compounds and is particularly useful for therapeutic uses of the compounds. Bading et al (2000) showed in rats that co-administration of eniluracil with 18F-FUra increased the radioactivity uptake into tumors and reduced interfering catabolites. Saleem et al (2000) reported similar findings in humans for eniluracil plus18F-FUra In Example 3, we demonstrate that eniluracil also enhances the DNA incorporations of BrUra, a 5-X-Ura compound, and reduces its circulating catabolites.

The DPDase inhibitors discussed are illustrative and any compound with similar inhibition of DPDase could be co-administered with the 5-X-Ura compounds, for either therapeutic or imaging purposes.

As another method of increasing the effective dose of 5-X-Ura compounds, the invention encompasses combining any of the diagnostic or therapeutic methods described with a prodrug that increases the intracellular supply of the co-substrate, deoxyribose-1-phosphate. The reverse reaction of TPase, is much slower in cells than the forward reaction. It appears that the rate limitation is the intracellular supply of the co-substrate for the reverse reaction, deoxyribose-1-phosphate, (dR-1-P). It is known that the reaction rate increases when a suitable precursor for dR-l-P is supplied. Such a precursor can be co-administered with 5-X-Ura compounds. The co-substrate prodrug can be selected from the group of dIno, dUrd, dThd, and other deoxyribonucleosides. Since dR-1-P does not cross cell membranes, its intracellular supply must be increased via a precursor. Deoxyribonucleosides are converted by the forward reaction of TPase or purine nucleoside phosphorylase into dR-1-P and its corresponding base. Thus, compounds such as dIno, dUrd, and dThd are all effective in cell culture at increasing the rate of the reverse reaction for TPase, see Examples 7 and 8. For either therapeutic or diagnostic uses, this increase in reaction rate produces increased intracellular trapping of the desired agent, with increased cytotoxicity or greater imaging sensitivity.

In a preferred embodiment, the co-substrate prodrug is deoxyinosine (dIno). Although any deoxyribonucleoside seems to be effective in cell culture, deoxyinosine has been shown to be effective in vivo. In a study in rats with tumors, co-administration of dIno with 5-FUra increased the intracellular concentrations of FdUrd-derived anabolites, indicating that dIno shifted intracellular metabolism towards preferential utilization of the TPase pathway, rather than UrdPase pathways Ciccolini et al (2000; 2001).

In another preferred embodiment, the co-substrate is thymidine (dThd). 5-F-Ura was given simultaneously with dThd in three separate clinical studies, reported by Kirkwood et al (1980), Au et al (1982), and Woodcock et al (1980). Each of these studies found conversion of 5-F-Ura to 5-F-dUrd when thymidine was co-administered, but not when 5-F-Ura was administered alone.

In another aspect the present invention encompasses inhibiting the action of TPase, in order to enhance the diagnostic utility of any positron-labeled 5-R-dUrd, shown below, particularly when R═H.

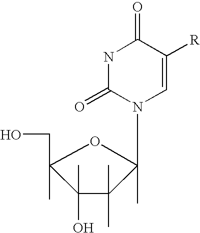

The result of inhibiting TPase is analogous to inhibiting DPDase. Inhibiting the catabolism of deoxyribonucleosides (produced by the forward reaction of TPase) provides the advantages of permitting greater imaging sensitivity or increased delivery of cytotoxic deoxyribonucleosides. Moreover, since Tease is considered to be an angiogenic agent, inhibition may confer a therapeutic benefit.

A variety of pyrimidine nucleosides (5-R-dUrd) have been proposed and/or tested as potential imaging agents for assessment of cellular proliferation, including [¹¹C]-dThd (thymidine), [¹²⁴I]-IdUrd (iododeoxyuridine), and [⁷⁶Br]-BrdUrd (bromodeoxyuridine). Although these probes are very efficient at labeling cells in culture, they have limited usefulness in patients due to rapid degradation by TPase to a variety of radiolabeled catabolites.

There have been attempts, to overcome rapid catabolism of these pyrimidine probes, by synthetic modification of the deoxyribose sugar. 3′-Fluoro-thymidine (FLT) and various 2′-arabino-fluorinated substituted pyrimidines (e.g., FMAU, FIAU, FBrAU, and FAUT) have been tried to overcome the rapid catabolism of unsubstituted versions. However, none of these modified pyrimidines are as efficient at labeling cells as the pyrimidines with the unmodified deoxyribose sugar.

The present invention overcomes this problem by means of temporarily inhibiting TPase during exposure to the pyrimidine-imaging probe. Co-administration of a TPase inhibitor with these pyrimidine PET imaging probes would increase their effectiveness. We estimate that 10- to 100-fold increase in effectiveness is possible, based upon increased yield of incorporation of the dose of radiolabel into DNA in tumors or other tissues of interest.

Several TPase inhibitors have been in development for direct therapeutic use. The present invention encompasses use of radiolabeled pyrimidines in combination with TPase inhibitors. In addition, various 5-substituted uracil derivatives (5-X-Ura), at clinically achievable concentrations, are effective inhibitors of TPase. Specific compounds include thymine, iodouracil, bromouracil, and trifluorothymine (see Example 9).

In a preferred embodiment, the present invention is used for the diagnosis and treatment of humans suffering from tumors, in particular, from cancerous tumors. However, it will be appreciated by persons of skill in the art that the invention can be used in other mammals, for example in veterinary applications, and in non-mammalian species having a similar biochemical/physiological basis for pathological conditions relating to angiogenesis that the present invention is intended to diagnose or treat.

### BRIEF DESCRIPTION OF THE FIGURES

FIG. 1 is a comparison of the association of survival and TPase levels in four different cancer types.

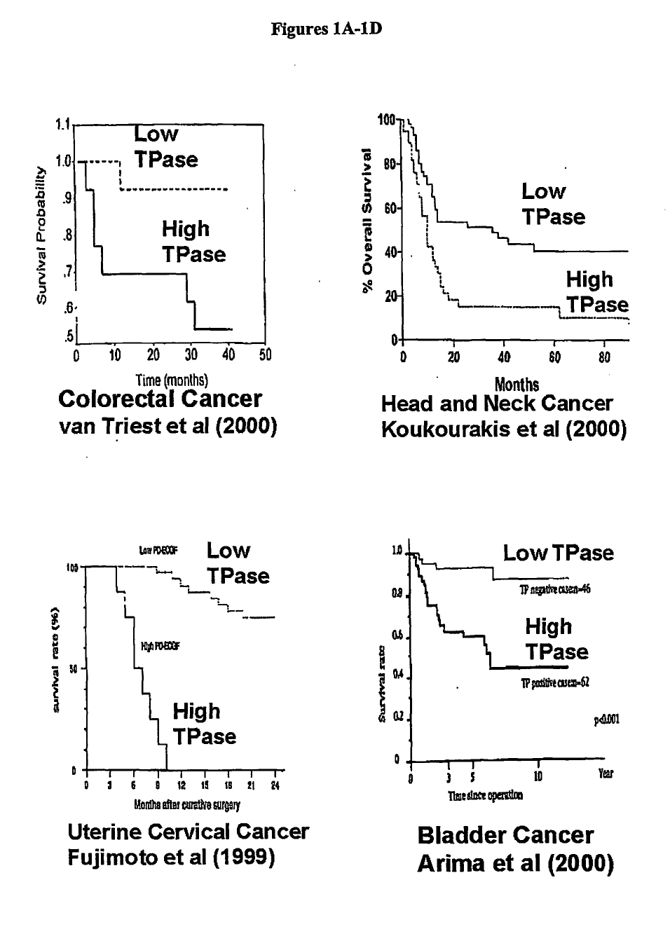

FIG. 2 is a comparison of 5-X-Ura incorporation into DNA in five cell lines.

FIG. 3 is a comparison of BrUra incorporation with different deoxyribonucleosides.

### DETAILED DESCRIPTION OF THE INVENTION

### EXAMPLE 1

### Synthesis of 5-X-Ura

Production of IUra and BrUra from Ura

A solution of 1,3,4,6-tetrachloro-3a,6a-diphenylglycouril, 0.5 mg/ml, was prepared in chloroform (all reagents are available from Sigma Chemical Co., St. Louis, Mo., unless otherwise noted). 0.5 ml (250 pg, 578 μmol) of solution was dried with a stream of nitrogen. 300 μl of Ura (2610 μmol, 8.7 mM in 0.25M potassium phosphate buffer, pH 7.5) was added and then 100 μl of NaI or NaBr (670 μmol, 6.7 mM in water) was added to the Ura. The mixture was heated at 60° C. for 15 min. Yields, based on halide, were 79% for lUra and 56% for BUra. At the end of the reaction, the reaction mixture was diluted with water and the products were measured by reverse-phase HPLC. The mobile phase was 20-50 mM acetic acid with 6-16% ACN.

This procedure works with small quantities of material, and is also suitable for labeling of Ura by therapeutic isotopes by substituting atoms such as [⁸²Br] or [¹²⁵-I] for the non-radioactive halides. Similarly, positron-labeled Ura can be generated by substituting atoms such as [⁷⁶-Br] or [¹²⁴-I]. Guenther et al. (1998) have shown that this procedure is suitable for use with radioisotopes; they prepared the nucleoside [¹²⁴-I]-dUrd, rather than the nucleobase, [¹²⁴-I]-Ura, but the quantities of materials were comparable.

[¹¹C]-Thymine is synthesized via published procedures, for example, Mankoff, 1998; Steel et al, 1999.

Similarly, [¹¹C]-Trifluoromethyl-thymine could be synthesized by these methods. [¹⁸F]CF₃-Ura could be produced via [¹⁸F]-trifluoromethyl iodide addition to Ura, by analogy with the standard procedure for methyl iodide.

The carbon atoms in positions 2,4,5, or 6 or any combination thereof of uracil can also be labeled with [¹¹C], prior to the addition of the 5-X-substituent. This is demonstrated for non-radioactive uracil. Chakraborty et al. (1997) have demonstrated the process for [¹¹C] in the 2-position of uracil, and its application for therapeutic isotopes could follow these procedures.

### EXAMPLE 2

### Conversion of Thymine Analogs Into Thymidine Analogs with Subsequent Incorporation Into DNA

Thymine and BrUra were used as representative of the 5-X-Ura class of compounds. These compounds were incubated for 24 hours with each of five human tumor cell lines that had been phenotyped for TPase activity by the method of Gan (1981) (cell lines can be obtained from ATCC, Manassas, Va.). In brief, cell extracts were incubated with thymidine, and the rate of formation of thymine was measured:

| Cell Type | TPase activity |
|---|---|
| CEM | <0.3 nmol/hr/mg protein |
| MOLT-4 | <0.3 |
| K-562 | <0.3 |
| RAJI | 2.0 |
| U937 | 24.9 |

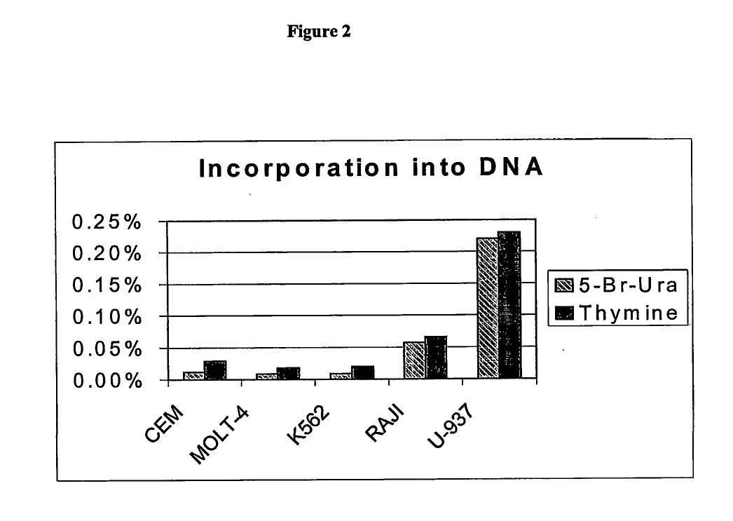

In the second part of this experiment, the same cell lines were incubated with BrUra (20 μM) and thymine (20 μM). FIG. 2 compares incorporation into DNA. The trends for BrUra and thymine were identical. The results of these studies are;

1. The 3 cell lines with lowest TPase activity (CEM, MOLT-4, and K562) also have the lowest levels of incorporation into DNA.

2. RAJI cells have intermediate TPase activity and intermediate DNA incorporation.

3. U-937 cells have the highest TPase activity and the greatest incorporation into DNA.

These data demonstrate that the accumulation of thymine analogs within tumor cells is related to the activity of TPase. Thus, radiolabeling a thymine analog with a positron emitter produces a probe to monitor the localization of TPase within the tumor and/or normal host tissue. The probe can be detected by use of imaging technologies such as a PET scanner without use of invasive procedures to take biopsies. The unlabeled thymine analog or the thymine analog labeled with a therapeutic isotope can also be used for therapy, since it is preferentially localized in tumors with high expression of TPase.

These data also demonstrate that thymine (and its analogs) are not simply probes for cellular proliferation. All of the above cells had identical proliferation rates (doubling times of about 24 hours), but the incorporation of thymine or bromouracil varied more than 10-fold, and was directly associated with the amount of TPase in each cell line.

### EXAMPLE 3A

### 5-X-Ura Incorporation Into DNA In Vivo

Dunning rat prostatic tumors were implanted (250,000 cells) subcutaneously in 3 rats. Two weeks post-implantation, the tumors had reached 7-20 g in size and the rats weighed about 250 g. At 2 weeks post-implantation, TPase was measured. BrUra was prepared in an aqueous solution, adjusted to pH 9 (the same formulation used clinically for its analog, FUra), and injected intravenously via a tail vein. The rats were sacrificed 105 minutes after the injection, and the tumors were harvested. DNA from the tumor was isolated, digested, and the nucleosides analyzed by HPLC (Klecker, 1994). BrdUrd was found in the DNA of the tumors, replacing 0.15% of the dThd bases. These results indicate that TPase in the tumor converted BrUra to BrdUrd, which was then “trapped” in DNA.

### EXAMPLE 3B

### Incorporation of 5-X-Ura Into DNA In Vivo, and Modulation with Eniluracil

U-937 cells (ATCC; Manassas, Va.) were implanted subcutaneously in nude mice. When tumors grew to about 0.5 gram, [³H]-BrUra was injected i.v. One set of mice received eniluracil, 2 mg/kg, injected i.p. thirty minutes prior to i.v. [3H]-BrUra, and the other set received no treatment prior to [³H]-BrUra i.v. Both sets of mice were killed 24 hours later, and tumors and blood removed. DNA from tumors was extracted and analyzed using our established techniques (Klecker et al., 1994). [³H]-BrdUrd was found in DNA in tumors by radiochromatography, indicating conversion of [³H]-BrUra in vivo, similar to our results for cell culture. Co-administration of eniluracil increased incorporation of radioactivity into DNA from [³H]-BrUra five-fold, and also reduced the circulating catabolites in plasma by at least 4-fold.

### EXAMPLE 4

### General Procedures for Clinical Use

The positron-labeled, antitumor probe is prepared shortly before use. That is, within 2 hours of injection for [¹¹C], within 4 hours of injection for [¹⁸F], within 24 hours of injection for [⁷⁶Br], or within one week for [¹²⁴⁻I]. A positron-labeled, antitumor probe with an activity of I to 300 mCi is injected into the patient as an intravenous bolus, i.e., within less than 5 minutes. The patient is placed in a PET scanner, and images are obtained, using standard technique at 5- to 10-minute intervals following the injection, up to at least 60 minutes, preferably to 90 minutes, when image quality is satisfactory. When 18F or another positron-emitting isotope with a longer half-life is used, imaging at longer intervals, hours or a day or more may be feasible. Variations and improvements in machine technology may permit even longer imaging periods, which is desirable. It is within the scope of the present invention for an operator skilled in the art of PET scanning to modify the method in such ways as changes in PET scans require or allow.

### EXAMPLE 5

### Use of Positron-Labeled Antitumor Probes for Selection of Chemotherapy

A patient with a tumor can be imaged with one or more of the positron-labeled antitumor probes according to the procedures in Example 4. One or more of the labeled probes is injected. If more than one antitumor probe is used, a 90 minute period (or longer) between injections is necessary for [¹¹C] labeled probes. Four or more hours between separate injections may be required if [¹⁸F] is used, or longer intervals when other isotopes with longer half-life are used.

Based upon the localization of the probe demonstrated in the images, therapy can be guided by whether the tumor has high or low TPase. If the tumor has high expression of TPase, then therapy with TPase inhibitors may be appropriate, or the use of 5-X-Ura to localize in DNA. Other chemo-therapeutic agents can also be evaluated. However, if there is low TPase activity, the tumor would be classified as resistant to 5-X-Ura therapy, and different treatments considered.

### EXAMPLE 6

### Evaluation of Modulators for Antitumor Drug Delivery to Tumors

The procedures in Example 4 can be used to obtain a baseline evaluation of probe localization within the tumor, in the absence of any modulation attempts. These procedures are then repeated in the presence of a specific modulation strategy, or a series of modulation attempts, and the images are compared to determine success or failure of the modulation.

### EXAMPLE 7

### Non-Radioactive 5-X-Ura Inhibits Tumor Growth

The five human-derived tumor cell lines shown below were incubated with BrUra at concentrations from 0.5 to 4.5 mM for 72 hours. (Cell lines are available at the ATCC, Manassas, Va.) Separate incubations were conducted in the presence or absence of 50 μM dUrd. Growth inhibition was determined as a percentage of control values for cells incubated without BrUra or dUrd. Consistent with the experiments described in Example 8, co-incubation with dUrd increased cytotoxicity of BrUra in all groups. As expected from their high level of TPase expression, U937 were the only cells with notable toxicity at 0.5 mM, and also the greatest toxicity at 1.5 and 4.5 mM. CEM, MOLT4, and K562 were substantially less affected by incubation with BrUra, consistent with a low intracellular activation rate because of low TPase expression. In the entire experiment, the only deviation from expected results was that the Raji cell line exhibited less toxicity than expected based upon its intermediate level of TPase expression.

% Growth Inhibition, Based Upon Control Doubling Time=24 Hr. BrUra Concentrations Ranged from 0.5 to 4.5 mM. Incubations were Conducted for 72 Hr in the Presence or Absence of 50 μM ddUrd.

| | Without dUrd | | | With dUrd | | |
|---|---|---|---|---|---|---|
| Cell Type | 0.5 mM | 1.5 mM | 4.5 mM | 0.5 mM | 1.5 mM | 4.5 mM |
| CEM | 6 | 23 | 89 | 13 | 65 | 95 |
| MOLT4 | 6 | 26 | 66 | 8 | 25 | 85 |
| Raji | 2 | 27 | 65 | 9 | 24 | 67 |
| U937 | 16 | 47 | 100 | 28 | 77 | 100 |
| K562 | −7 | 13 | 57 | −6 | 28 | 77 |

### EXAMPLE 8

### Enhancement by of 5-X-Ura Trapping into DNA by dR-1-P Prodrugs

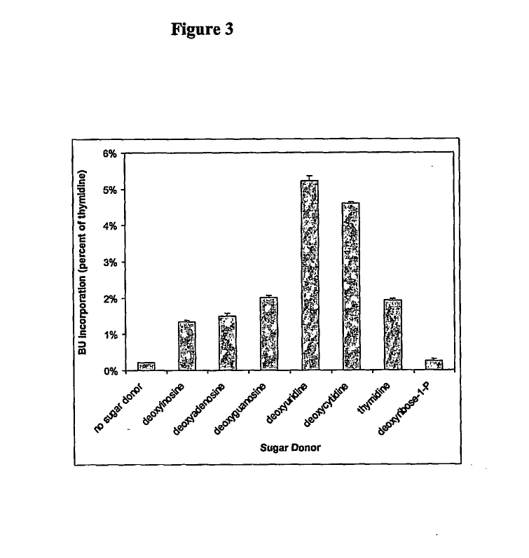

The effect of dR-1-P prodrugs was examined in U-937 cells. Cells were incubated for 24 hours with 20 μM BrUra and 20 μM of six different deoxyribonucleosides. The use of prodrugs for dR-1-P is described as a way of driving the reaction catalyzed by TPase in the direction of thymidine production. As illustrated in the bar chart, in FIG. 3, the maximum enhancement of BrUra trapping in DNA was 24-fold, for dUrd. An increase of 6-fold was found for dIno.

### EXAMPLE 9

### 5-X-Ura Inhibits TPase

The inhibition of TPase by 5-X-Ura was demonstrated in separate experiments that used (A) intact human hepatocytes, or (B) intact tumor cells co-incubated with cytosol from human hepatocytes.

Cryopreserved human hepatocytes were procured from In Vitro Technologies, Inc. (Catonsville, Md.) and processed according to the supplier's instructions. TPase activity was monitored for one hour by measuring formation of [³H]-Ura from [³H]-dUrd, 0.2 μM. Co-incubation of tritiated dUrd with 100 μM BrUra inhibited TPase by 48%, and 500 μM of BrUra inhibited TPase by 85%.

[³H]-dThd (0.02 μM) or [³H]-dUrd (0.1 μM) was incubated with either U-937 or MOLT-4 tumor cells, and incorporation of [³ M-dThd into DNA was measured after 3 hours. Cytosol (S100) was prepared from human liver by differential centrifugation. This cytosol, which contains TPase, was co-incubated with U-937 or MOLT4 tumor cells to simulate the degradation of pyrimidine nucleosides in the body. Incorporation of [³H]-dThd into DNA was decreased to 5-35% of baseline levels. When BrUra (500 μM) was also added to the incubation mixture, TPase inhibition was observed as increased incorporation of [³H]-dThd in DNA, back to 39-89% of baseline levels.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

Referenced cited herein are listed below for convenience and are incorporated by reference.

References

Arima J, Imazono Y, Takebayashi Y, et al. Expression of thymidine phosphorylase as an indicator of poor prognosis for patients with transitional cell carcinoma of the bladder. Cancer 2000; 88(5):1131-8.

Au J L, Rustum Y M, Ledesma E J, Mittelman A, Creaven P J. Clinical pharmacological studies of concurrent infusion of 5-fluorouracil and thyridine in treatment of colorectal carcinomas. Cancer Res 1982;42:2930-7.

Bading J R, Alauddin M M, Fissekis J D, Shahinian A H, Joung J, Spector T, Conti P S. Blocking catabolism with eniluracil enhances PET studies of 5-[¹⁸F]fluorouracil pharmacokinetics. J Nucl Med 2000; 41:1714-24.

Baker S D, Diasio R B, O'Reilly S, et al. Phase I and pharmacologic study of oral fluorouracil on a chronic daily schedule in combination with the dihydropyrimidine dehydrogenase inactivator eniluracil. J Clin Oncol. February 2000;18(4):915-26.

Chakraborty P K, Mangner T J, Chugani H T. The synthesis of no-carrier-added [¹¹C]urea from [¹¹C]carbon dioxide and application to [¹¹C]uracil synthesis. Appl Radiat Isot 1997; 48:619-21.

Ciccolini J, Cuq P, Evrard A, Giacometti S, Pelegrin A, Aubert C, Cano J-P, Iliadis A. Combination of thymidine phosphorylase gene transfer and deoxyinosine treatment greatly enhances 5-fluorouracil antitumor activity in vitro and in vivo. Mol Cancer Ther 2001; 1: 133-139.

Ciccolini J, Peillard L, Evrard A, Cuq P, Aubert C, Pelegrin A, Pormento P, Milano G, Catlin J. Enhanced antitumor activity of 5-fluorouracil in combination with 2′-deoxyinosine in human colorectal cell lines and human colon tumor xenografts. Clin Cancer Res 2000; 6:1529-35.

Endo M, Shinbori N, Fukase Y, Sawada N, et al. Induction of thymidine phosphorylase expression and enhancement of efficacy of capecitabine or 5′-deoxy-5-fluorouridine by cyclophosphamide in mammary tumor models. Int J Cancer Sep. 24, 1999;83(1):127-34.

Fujimoto J, Sakaguchi H, Aoki I, Tamaya T. The value of platelet-derived endothelial cell growth factor as a novel predictor of advancement of uterine cervical cancers. Cancer Res. Jul. 1, 1999;60(13):3662-5.

Fukushima M, Suzuki N, Emura T, et al. Structure and activity of specific inhibitors of thymidine phosphorylase to potentiate the function of antitumor 2′-deoxyribonucleosides. Biochem Pharmacol. May 15, 2000;59(10):1227-36.

Gan T E, Hallam L, Pilkington G R, et al. A rapid and simple radiometric assay for thymidine phosphorylase of human peripheral blood cells. Clinical Chimica Acta 1981; 116:231-6.

Guenther L Wyer L, Knust E J, Finn R D, Koziorowski J, Weinreich R. Radiosynthesis and quality assurance of 5-[¹²⁴I]iodo-2′-deoxyuridine for functional PET imaging of cell proliferation. Nuclear Med Biol 1998; 25:359-65.

Hamada A, Fukushima S, Morinaga A, Saneyoshi M, Kawaguchi T, Nakano M. Differential effects of acyclothymidine, a potent pyrimidine nucleoside phosphorylase inhibitor, on the pharmacokinetics of doxifluridine in rabbits via oral administration. Biol Pharm Bull. 1993; 16:1297-300.

Ishikawa T, Sekiguchi F, Fukase Y, Sawada N, Ishitsuka H. Positive correlation between the efficacy of capecitabine and doxifluridine and the ratio of thymidine phosphorylase to dihydropyrimidine dehydrogenase activities in tumors in human cancer xenografts. Cancer Res Feb. 15, 1998;58(4):685-90.

Kirkwood J M, Ensminger W, Rosowsky A, Papathanasopoulos N, Frei E 3rd. Comparison of pharmacokinetics of 5-fluorouracil and 5-fluorouracil with concurrent thymidine infusions in a Phase I trial. Cancer Res. 1980;40:107-13.

Klecker R W, Jr., Collins J M. Thymidine phosphorylase as a target for imaging and therapy with thymine analogs. Cancer Chemother Pharmacol 2001;48:407-12.

Klecker R W, Katid A G, Collins J M. Toxicity, metabolism, DNA incorporation with lack of repair, and lactate production of 1-(2′-fluoro-2′-deoxy-B-D-arabinofuranosyl)-5-iodouracil (FIAU) in U-937 and MOLT-4 cells. Mol Pharmacol 1994; 46:1204-1209.

Koukourakis M I, Giatromanolaki A, Fountzilas G, et al. Angiogenesis, thymidine phosphorylase, and resistance of squamous cell head and neck cancer to cytotoxic and radiation therapy. Clin Cancer Res. February 2000 ;6(2):381-9.

Makower D, Wadler S, Haynes H, Schwartz E L. Interferon induces-thymidine phosphorylase/platelet-derived endothelial cell growth factor expression in vivo. Clin Cancer Res 1997; 3:923-9.

Mankoff D A, Shields A F, Link J M, Graham M M, Muzi M, Peterson L M, Eary J F, Krohn K A Kinetic analysis of 2-[11C]thymidine PET imaging studies: validation studies. J Nucl Med 40:614-24, 1999.

Matthes E, Barwolff D, Langen P. Inhibition by 6-aminothymine of the degradation of nucleosides (5-iododeoxyuridine, thymidine) and pyrimidine bases (5-iodouracil, uracil and 5-fluorouracil) in vivo. Acta Biol Med Ger. 1974; 32:483-502.

Saleem A, Yap J, Osman S, Brady F, Suttle B, Lucas S V, Jones T, Price P M, and Aboagye EO (2000). Modulation of fluorouracil tissue pharmacokinetics by eniluracil: in-vivo imaging of drug action. Lancet 355:2125.

Sawada N, Ishikawa T, Fukase Y, et al. Induction of thymidine phosphorylase activity and enhancement of capecitabine efficacy by taxol/taxotere in human cancer xenografts. Clin Cancer Res April 1998;4(4):1013-9.

Sawada N, Ishikawa T, Sekiguchi F, Tanaka Y, Ishitsuka H. X-ray irradiation induces thymidine phosphorylase and enhances the efficacy of capecitabine (Xeloda) in human cancer xenografts. Clin Cancer Res October 1999;5(10):2948-53.

Schuller J, Cassidy J, Dumont E, Roos B, Durston S, Banken L, Utoh M, MoriK, Weidekamm E, and Reigner B (2000). Preferential activation of capecitabine in tumor following oral administration to colorectal cancer patients. Cancer Chemother Pharmacol. 45:291-7.

Shimma N, Umeda I, Arasaki M, et al. The design and synthesis of a new tumor-selective fluoropyrimidine carbamate, capecitabine. Bioorg Med Chem July 2000;8(7):1697-706.

Steel C J, Brady F, Luthra S K, Brown G, Khan I, Poole K G, Sergis A, Jones T, Price P M. An automated radiosynthesis of 2-[¹¹C]thymidine using anhydrous [¹¹C]urea derived from [¹¹C]phosgene. Appl Radiat Isot 1999; 51:377-88.

Usuki K, Saras J, Waltenberger J, et al. Platelet-derived endothelial cell growth factor has thymidine phosphorylase activity. Biochem Biophys Res Commun. May 15, 1992;184(3):1311-6.

Vaidyanathan G, Larsen R H, Zalutsky M R. 5-[²¹¹At]Astato-2′-deoxyuridine, an alpha-particle-emitting endoradiotherapeutic agent undergoing DNA incorporation. Cancer Res 1996; 56:12049.

van Triest B, Pinedo H M, Blaauwgeers J L et al. Prognostic role of thymidylate synthase, thymidine phosphorylase/platelet-derived endothelial cell growth factor, and proliferation markers in colorectal cancer. Clin Cancer Res. March 2000;6(3): 1063-72.

Woodcock T M, Martin D S, Damin L A, Kemeny N E, Young C W. Combination clinical trials with thymidine and fluorouracil: a phase I and clinical pharmacologic evaluation. Cancer 1980; 45(5 Suppl):1135-43.

We claim:

1. A 5-X-Ura compound having the formula: 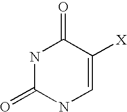
wherein:
(a) X is selected from the group consisting of CH₃, ¹¹C—CH₃, CF₃ , ¹⁸F—CF₃, Br, ⁷⁶Br, I, and ¹²⁴I, and
(b) at least one of X or one of the pyrimidine carbons, C-2, 4, 5, and 6, is a positron emitter.

2. A pharmaceutical composition comprising the compound of claim 1 and an inert carrier.

3. The pharmaceutical composition of claim 2, further comprising a DPDase inhibitor.

4. The pharmaceutical composition of claim 2, further comprising a prodrug that increases the intracellular supply of the co-substrate, deoxyribose-1-phosphate so as to increase the rate of transformation of the compound.

5. The pharmaceutical composition of claim 4, wherein the prodrug is selected from the group consisting of dIno, dUrd, and dThd.

6. The pharmaceutical composition of claim 5, wherein the prodrug is dIno.

7. The pharmaceutical composition of claim 5 wherein the prodrug is dThd.

8. A method of detecting TPase levels in a patient comprising
(a) administering the positron-labeled compound of claim 1 to the patient; and
(b) scanning all or part of the patient with a PET scanner so as to determine levels of TPase activity.

9. The method of claim 8 wherein the patient is a mammal.

10. The method of claim 9 wherein the patient is a human.

11. The method of claim 8, wherein the patient has a tumor.

12. The method of claim 8, wherein the patient has received treatment with a drug that increases or decreases TPase levels.

13. The method of claim 12, wherein the drug is an inhibitor or an inducer of TPase.

14. The method of claim 8, wherein the patient has or is suspected of having increased angiogenesis.

15. The method of claim 8, wherein the patient is receiving a treatment for tumors.

16. A method of treating a patient who has, or is suspected of having, elevated TPase levels, comprising administering a therapeutically effective amount of the compound of claim 1.

17. The method of claim 16 wherein the patient is a mammal.

18. The method of claim 17 wherein the patient is human.

19. The method of claim 16, wherein the compound of claim 1 contains as X a therapeutic isotope.

20. The method of claim 19, wherein the therapeutic isotope is one of the group consisting of alpha and beta emitters.

21. The method of claim 19, wherein the isotope is selected from the group consisting of [²¹¹At], [¹²⁵I], [¹³¹I], and [⁸²Br].

22. The method of claim 16, further comprising the step of co-administering a prodrug for deoxyribose-1-phosphate.

23. The method of claim 16, further comprising the step of co-administering a DPDase inhibitor.

24. The method of claim 16, further comprising the step of co-administering a TPase inhibitor.

25. A pharmaceutical composition comprising a positron-labeled 5-R-dUrd, having the formula: 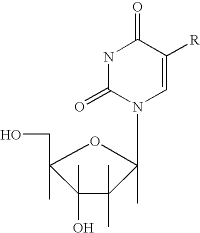
wherein:
(a) R is selected from the group consisting of H, CH₃ , ¹¹C—CH₃, CF₃, ¹⁸F—CF₃, Br, ⁷⁶Br, I, and ¹²⁴I;
(b) at least one of the atoms of R or one of the pyrimidine carbons, C-2, 4, 5, or 6 or deoxyribose carbons, C-1′, 2′, 3′, 4′, or 5′ is a positron emitter; and
(c) said composition co-administered with a TPase inhibitor in an amount sufficient to enhance the diagnostic utility of the composition.

26. A method of synthesizing the composition of claim 1, comprising the steps of
(a) providing 1,3,4,6-tetrachloro-3α,6α-diphenylglycouril as a dried residue,
(b) combining a solution of uracil with the 1,3,4,6-tetrachloro-3α,6α-diphenylglycouril,
(c) combining the solution of step (b) with a halogen salt, and
(d) allowing the reaction to proceed.
